(19) United States
(12) Patent Application Publication
(10) Pub. No.: US 2010/0017226 A1
(43) Pub. Date: Jan. 21, 2010

(54) MEDICAL WORKFLOW ONCOLOGY TASK ASSISTANCE

(75) Inventors: Manivannan Sundarapandian, Bangalore (IN); Vijaya Ramanathan, Ghorpuni Pune (IN)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc., Malvern, PA (US)

(21) Appl. No.: 12/175,990

(22) Filed: Jul. 18, 2008

(51) Int. Cl. G06F 19/00; G06F 9/46; G06Q 50/00

(52) U.S. Cl. 705/3; 705/9; 705/2

(57) ABSTRACT

Assistance is provided in an oncology workflow. A tool or assistance link is provided as part of the workflow or display of task information. Task information for other patients may assist the medical professional in current task decisions. For example, the target dose, warning dose, fraction dose and number of fractions for patients with tumors at a same site (e.g., brain), with a similar size (e.g., 1 cm), a similar stage (e.g., stage 2), and/or other diagnostic information may be useful in a prescription generation task. While completing the task, the user activates the tool or assistant to determine what has been done in similar circumstances. The tool or assistances link acts as colleague advice for determining a current treatment.

### BACKGROUND

The present embodiments relate to medical workflow assistance. For example, medical professionals are assisted in oncology tasks.

In oncology, users identify cancerous regions for treatment, such as contouring in an image. An oncologist or dosimetrist determines a prescription for treating the tumor. A dosimetrist creates a plan to implement the prescription. A therapist positions and verifies positioning for performance of the treatment. A physicist may test or check the plan or the therapy equipment. Other medical professionals may have other tasks. The oncologists, dosimetrists, therapists, physicists or others may perform different ones of the tasks and/or verify proper completion of the tasks by others.

To assist the medical professionals in completing tasks, workstations may be provided. In some oncology departments, there may be multiple networked workstations for various medical professionals. However, the workstations are limited to assisting specific tasks for particular patients, such as a computer assisted diagnosis program providing a second opinion for identifying tumors from an image.

The medical professionals complete the tasks for each patient. The medical professional makes a decision in terms of prescription or treatment protocol on a case-by-case basis. The treatment course is determined based on the patient's clinical record. However, as in many tasks, an individual's knowledge and judgment for the treatment course may be based on limited information.

### BRIEF SUMMARY

By way of introduction, the preferred embodiments described below include methods, systems, computer readable media, and instructions for assisting an oncology or other medical workflow. A tool or assistance link is provided as part of the workflow or display of task information. Task information for other patients may assist the medical professional in current task decisions. For example, the target dose, warning dose, fraction dose and number of fractions for patients with tumors at a same site (e.g., brain), with a similar size (e.g., 1 cm), a similar stage (e.g., stage 2), and/or other diagnostic information may be useful in a prescription generation task. While completing the task, the user activates the tool or assistant to determine what has been done in similar circumstances. The tool or assistant link acts as colleague advice for determining a current treatment.

In a first aspect, a method is provided for assisting an oncology workflow. A user interface is displayed for each of a plurality of oncology tasks. The user interface for each of the oncology tasks includes one or more first tools for receiving input for a variable of the oncology task. User selection of an assistant tool is received for at least a first oncology task of the oncology tasks. Information is displayed for the first oncology task in response to the user selection. The information includes a list of values for the variable and use frequency for each value of the list and is from records of a plurality of patients.

In a second aspect, a system for assisting an oncology workflow is provided. A processor connects with a user input and a display. The processor is operable to receive user selections of values for respective variables associated with a prescription generation oncology task, plan generation oncology task, patient positioning oncology task, imaging selection oncology task, physicist plan quality assurance (QA) oncology task, or combinations thereof. The processor is operable to output to the display example values, corresponding to the oncology task, used for other patients with successful outcomes and having similar diagnostic information as a current patient. The display is operable to display the example values.

In a third aspect, a computer readable storage medium has stored therein data representing instructions executable by a programmed processor for providing historical values in a medical workflow. The storage medium includes instructions for outputting statistical information for possible values in a current medical task as a function of past use of the possible values for other patients, and receiving user input of an actual value to be used for a current patient.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for assisting in an oncology workflow;

FIG. 2 is a graphical representation of one embodiment of an oncology workflow user interface;

FIG. 3 is a graphical representation of one embodiment of an oncology task user interface with an assistant tool;

FIG. 4 is a graphical representation of one embodiment of an oncology task user interface with assistance information; and

FIG. 5 is a block diagram of one embodiment of a system for assisting in an oncology workflow.

### DETAILED DESCRIPTION OF THE DRAWINGS AND PRESENTLY PREFERRED EMBODIMENTS

Historical information assistance (ranked as per order of usage) is provided in medical workflow. In one embodiment, centralized data management and workflow for or between users is provided. For example, a workflow engine is provided for oncology specific use cases. Prescription generation, plan generation, patient positioning, type of imaging, plan QA, and/or other tasks are implemented to assist different oncology department users in their respective roles in treating a patient. Workstations communicate with a centralized data repository through DICOM governed standards or other database system. In other embodiments, different users operate with task specific information without centralization.

Regardless of the type of workflow engines, the medical professional has information for the specific patient to be treated. For example, when a clinical user marks (e.g., contours) a patient tumor, the user does so with only personal memory of historical or past trends of clinical treatment information. To improve workflow, a workflow assistant is provided. The values for variables of one or more tasks for other patients are indicated to the medical professional. The values are associated with statistical information, such as number of uses and success or failure associated with different value combinations. The medical professional may use this historical information to come up with a desired prescription or plan for the current patient. The historical information reflects group expertise given a particular diagnostic situation (e.g., brain tumor of a particular type and stage).

Data in a computerized patient record system provides a source for the historical information. The historical information assists medical professionals for more rapid task completion and/or more likely successful treatment decisions. In one embodiment, the workflow task assistant tool presents the clinical user access to historical data while planning or for completing oncology tasks. The tool may be made available through a single click or other activation.

The tool is specific to the task. Different clinical users, such as oncologists, dosimetrist, therapist, physicists or others are presented with information, which is relevant to the type of task performed by the user. For every task entry, a link may be provided to invoke the workflow task assistant tool. The historical or statistical information provided to the clinician is context specific. The type of information provided to the clinician is task centric. Once the tool is invoked, the user is able to see this information while attending to the task. To allow entry of a value for a current patient, the historical information may be hidden and unhidden.

Providing task specific statistical information allows the user to query for information across patients. The query may be for a centralized or distributed patient record system. The clinical user may be aided in implementing a current workflow task with the help of the historical clinical data, increasing efficiency in the workflow. The user may retrieve relevant information through a simple click or other navigation without having to run any queries or use search filters in a manual attempt to mine for information.

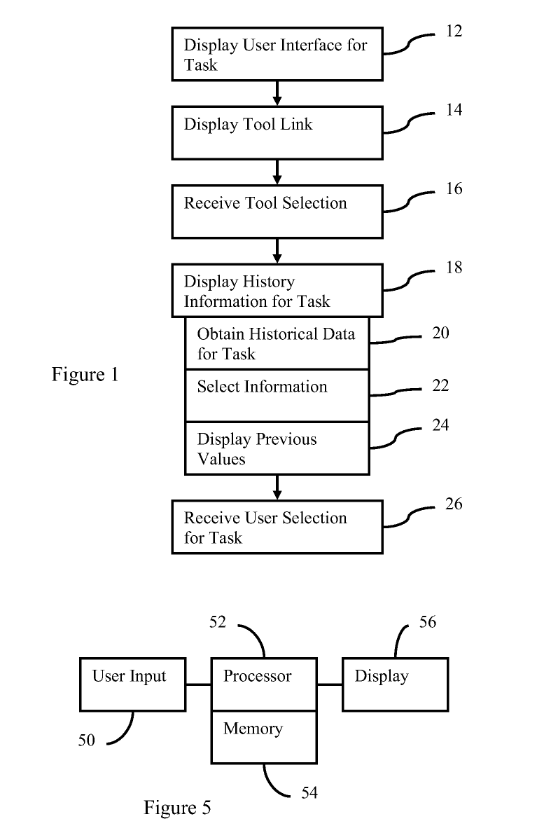

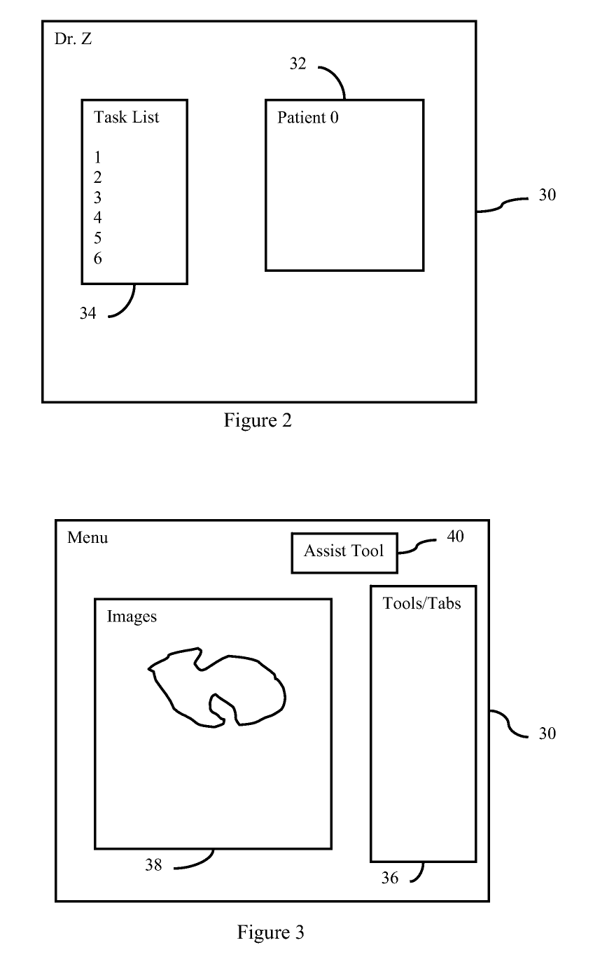

FIG. 1 shows a method for assisting an oncology workflow. The method is implemented with the user interfaces of FIGS. 2-4 or other user interfaces. The method is implemented by the system of FIG. 5 or another system. The acts of the method are performed in the order shown or a different order. Additional, different, or fewer acts may be provided. For example, acts 14 and/or 26 are optional. As another example, the tool for statistical information is provided outside of the context of a task-based user interface of acts 12 or 26.

In act 12, a user interface for a medical task is displayed. Any user interface may be displayed. The user interface may include information for a user, such as images, data, identifiers, or other information. The user interface includes components that may be selected, activated or used by a user to input one or more values. For example, graphic buttons, text entry boxes, selectable items or check boxes, menus, drop down menus, or other components are provided. The user interface allows a user to input information with user input devices, such as a trackball, keyboard, buttons, sliders, mouse, or other devices. Any user interface engine may be used, such as a browser user interface (e.g., Internet Explorer).

In one embodiment, the user interface is displayed for each of a plurality of tasks. Separate screens are provided for different tasks. One screen or display may include components or information for multiple tasks. In an oncology example, FIGS. 2 and 3 show two user interfaces 30. FIG. 2 shows a home or initial workflow user interface 30. The user interface 30 is for a specific medical professional (e.g., Dr. Z) or for multiple users. The user interface 30 includes a list 34 of tasks to be completed and information 32 about a specific patient. Other formats may be used. The list 34 may be for a specific patient or may be for the medical professional. The information 32 may be for multiple patients or a patient associated with a selected task from the list 34. The user interface 30 may not include the task list 34, instead bringing up tasks as performed or in response to selection of other information (e.g., selection of a patient from a list). The user interface 30 may not include the patient information 32. Other information may be provided, such as tabs for different types of tasks, menus, tools, or other components for user interaction.

The user interface 30, including the task list 34 and/or patient information 32, may be specific to the medical professional. For example, if Dr. Z is a therapist, then the task list 34 includes tasks related to Dr. Z's role (e.g., patient positioning tasks). The patient information 32 or patients listed may be tailored to Dr. Z's role as well (e.g., patients for which Dr. Z is expected to perform a task). The medical professional logs onto the system, and the software identifies the role, user and/or associated patients for the medical professional based on the login information. As new patients and tasks are assigned, data is entered to identify the responsible medical professionals. The user interface 30 is updated to include the new patient information.

FIG. 3 shows the user interface 30 for a particular oncology task. For example, the medical professional selects a task from the list 34 of FIG. 2 to navigate to the user interface 30 of FIG. 3. In the example shown, the task is for plan generation. An image or images 38 showing contours of identified tumors are displayed.

Different tools, tabs, or other components 36 are provided on the user interface 30 for the task. The components 36 allow entry of task specific information. One or more variables are associated with each task. In the plan generation example, the variables may include the type of plan (e.g., Block, Conformal, or Intensity Modulated Radiation Therapy (IMRT)), treatment modality (e.g., x-ray or electron), and/or treatment accessories (e.g., beam shaping devices and/or settings). Other variables may be included. Other possible options for one or more of the variables may be included. For example, different facilities may have different equipment, resulting in a different list of treatment accessories.

The components 36 allow selection or entry of values for the variables. For example, a menu structure, checkboxes, and/or text entry with or without a drop down list is provided to select a value of a variable. The components 36 are tools for receiving a value for the variable of the task. For example in plan generation, the Dosimetrist, Dr. Z, is responsible for plan creation. The components 36 include different boxes of information for inputting a selection of different values of variables for the task. Dr. Z selects the desired plan, aided in part by display of the images 38 and/or other patient specific information. Dr. Z may enter “IMRT” using “x-rays” “without any accessories” as the three values for the plan type, modality, and accessory variables.

Other examples include user interfaces 30 for prescription generation, patient positioning, type of imaging, and/or plan QA tasks. For a prescription generation task display of a user interface, the variables may include target dose, warning dose, fraction dose or number of fractions. After a patient tumor is contoured or otherwise identified, a treatment prescription is determined. For example, an “Inverse Planning” task is provided. An oncologist may perform the prescription generation task.

For a patient positioning task display of a user interface, the variables may include tolerance and/or image filters. Different treatment sites may have different tolerances. For example, a tumor close to organ at risk (e.g., the eye) may have very small tolerances as compared to the lungs. Different facilities, medical professionals, geographic regions, and/or equipment may have different tolerances even for a same treatment site. For positioning, the patient and/or tumor is imaged to verify proper position relative to the treatment devices. The patient normally undergoes a position verification session before the actual treatment occurs. During this session, the patient position is captured through a portal imager. The portal image is compared with a reference image in the treatment plan. If the portal image is of bad quality (e.g., artifacts), the user has the option of correcting or enhancing the image with the use of an appropriate filter or combination of filters. The images may be difficult to interpret. Different filters or combinations of filters may be applied to the images to assist in highlighting the tumor and/or landmarks for position alignment. A therapist may perform the patient positioning task (image acquisition, analysis of portal and reference images), while the Oncologist may perform image approval based on the results from Therapist.

For a type of imaging task display of a user interface, the variables may include the types of imaging, different image processing, different scaling, different warping, different filtering, and/or different rendering technique. Patient positioning may be performed with a 2D or 3D verification protocol. This is decided before the review session based on the treatment site and plan used. A therapist or oncologist may perform the patient positioning.

For a physicist check task display of a user interface, the variables may include type of phantom, test technique (QA Plans), and/or test sequence. The physicist verifies performance of the equipment for a specific patient and plan or in general.

Other user interfaces for other tasks may be provided. Other medical professionals may perform any one or more of the tasks. Different medical professionals may perform the same task, such as an oncologist verifying the task work of a dosimetrist. Different, fewer, or additional variables and/or available values for each variable may be provided. The user interface 30 includes patient specific information for performing the task and components for selecting values of variables to complete the task. In alternative embodiments, the patient specific information and/or components are provided separately or manually, such as in a written record.

In act 14, an assistant icon 40 is displayed. The assistant icon 40 is a selectable button, but may be another component (e.g., selected through menu navigation). FIG. 3 shows the assistant icon 40 displayed on the user interface 30 for a specific task. The assistant icon 40 may be displayed on any user interface 30. The same or different icon 40 is provided for different tasks. For example, as an assistant for activation, the same icon 40 is provided for any task display or other display associated with determining values for variables. The assistant icon 40 is part of a user input device in other embodiments. For example, a function key, dedicated button, or other input device activates the assistant function. In alternative embodiments, a separate assistant icon 40 is not provided, but the historical or statistical information is instead provided without user solicitation. For example, the historic values of different variables are displayed next to the variable, available values, and/or elsewhere on the user interface 30 without further activation.

In act 16, user selection of the assistant tool or icon 40 is received. Any user indication of a request for historical or statistical data may be used. For example, the user depresses a button or clicks on the icon 40. The resulting signal is generated and received as an indication of a request for assistance in determining values for the variables. The context of the selection may be used, such as the currently displayed or selected task or variable being received to identify the data to be provided. The assistant tool or icon 40 may be activated for any of one or more tasks.

In act 18, historical information responsive to the user selection is displayed. Alternatively, the historical information is displayed without specific request. The historical information is specific to the task, but may include general information. To display the information, data is obtained in act 20, specific data is selected in act 22, and the values associated with the historical information are displayed in act 24. Acts 20 and 22 may be combined into one act. Any data mining or database querying may be used to provide values used for other patients.

In act 20, historical or related information is obtained. The information is obtained as a function of the associated task. The task indicates the variables for which sample values are to be obtained. The values for each variable associated with the task, a subset of variables and/or variables associated with other tasks are obtained.

Other information may be obtained. For example, outcome information is obtained. The outcome may be based on a standard or guideline, set by an administrator or otherwise determined. For example, survival for the average or other amount of time for a particular type of cancer after treatment indicates a successful outcome. Other outcome criteria may be used, such as reoccurrence. No reoccurrence in a particular period indicates a desired outcome. The outcome information may be used to include data from patients with a desired outcome and exclude data from patients with an undesired outcome. The data presented to the user indicates approaches that have been successful in the past. Data may be presented regardless of outcome, such as where outcome data is not available, to highlight differences between successful and unsuccessful situations, or to indicate the success rate of a given value set where some are successful and others are not.

The records used for obtaining data may be in one location or many locations. For example, the records and stored values are for patients associated with a same medical institution. The records are maintained in a centralized or distributed database. By obtaining data for a medical institution, the local or institute-based practice information is provided. This provides values specific to the available equipment, training standards, administration requirements, or other criteria. A medical institution may include one clinic, a hospital, a group of physicians in a medical practice, a chain of hospitals, an insurance group, or other organization. In other embodiments, records from multiple different institutions are used. Either through data sharing, clinical study, or third party data management, access to data for patients at multiple institutes may be available. The data may be culled to represent treatment options available at a particular location.

The values are obtained from patient records. For example, the patient records are stored as a structured database where particular fields are provided for each variable. For example, patient data, such as the values for variables entered in act 26, is stored in data fields defined for each variable. This is done for all or some of the patients. The database storing the data for the data fields is accessed and queried for the desired information. For example, a query obtains all the patient records with values entered for the desired variables. In alternative embodiments, other data mining, such as from free text, identifies the values for variables.

In one embodiment, data is obtained by querying context specific information from the database in response to activation of the assistance tool. When the user tries to access information, the system generates a query based on the inclusive and exclusive information given the context. For example, the query requires a particular outcome value or range of values (e.g., no reoccurrence after 2 or more years) for patients with a treatment site, size or size range, and stage or range of stages of a tumor. The context of the task and current patient information determines the query or search to be used. The query may be generated based on the request and/or a table of task specific queries may be stored for selection.

In another embodiment, the data is used to update the historical information as the data is created. When a user completes a task, the context specific information to be used by the assistant tool is stored. This data is stored in a different set of tables than used for the patient records. When the user queries for task specific information, these tables are accessed for the required information. The tables are updated as data becomes available and any statistics updated as data becomes available. The historical or statistical information may be accessed more rapidly by providing data from the table representing the query. This may avoid accessing multiple tables or patient records and searching unwanted information.

In act 22, the data is obtained by selecting information from other patients with similar diagnostics as the current patient. The obtained data is obtained based on a same or sufficiently similar situation. Sufficiently similar may be a mathematical correlation or other measure of similarity meeting a threshold. For example, data for other patients that were treated for a brain tumor is obtained for assistance in determining the treatment for a current patient with a brain tumor. Data from patients with lung cancer is not used.

Any diagnostic information may be used for the selection. Diagnostic information includes treatment site (e.g., lungs), size of tumor, stage of cancer, age of patient, distribution of the tumor, sex of the patient, whether the patient is a smoker, and/or other variable that may affect treatment choices. The diagnostic information may be used in any combination. For example, only size, stage, and site are considered. Any logic for use of the variables may be provided, such as accounting for a range within each variable (e.g., patient of 30-40 years of age), matching fewer than all variables (i.e., select data for patients with three out of four variables with the same values), or providing a certain number of the closest or most similar cases.

The diagnostic information used for selection may vary depending on the task. For example, a prescription generation task is being performed. The data is selected for other patients with at least a corresponding size and stage of tumor as a current patient for which the prescription generation task is being performed. As another example, a prescription generation task is being performed. The data is selected for other patients with at least a corresponding treatment site. Variables from a current task or other tasks for the current patient may be used. For example, data for patients with a corresponding treatment site and type of treatment (modality, plan type, dose, fraction, and/or others) is selected.

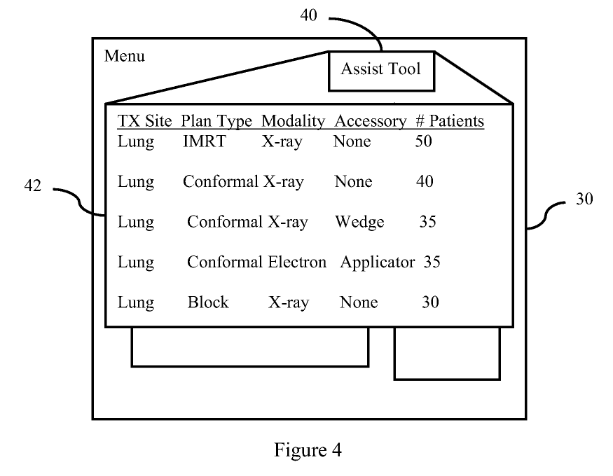

In act 24, the historical and/or statistical information is output. The actual values of variables for completing the task are output. For example, FIG. 4 shows the output for assistance in the plan generation task. The plan type, modality and accessory or accessory settings are output to show the values used. These actually used values indicate possible values to be used for a current patient and corresponding medical task.

Statistical information may be output. The number of times a particular value or combination of values was used is output. The number of patients associated with each value combination indicates, statistically, a weighting of previous approaches. FIG. 4 shows output of the frequency of use stat. Other statistics may be included, such as statistics based on outcome. An order of the list of values or combination may indicate most common use, providing statistical information to the user. Other values used to measure similarity and/or a statistic on degree of similarity may be output.

FIG. 4 shows outputting the statistical information and possible values for each of a plurality of variables associated with the current medical task, but information for other tasks or for fewer than all the variables for a given task may be output. Other information may be output, such as showing combinations of values associated with undesired results.

The number of uses indicates a use frequency for each value and/or combination of values for different variables. For example, the medical professional invokes the assistant tool in an ‘Inverse Planning task.’ Prescription information of other patients for the same treatment site or sites is presented. Details regarding ‘Target dose’, ‘Warning dose’, ‘Fraction Dose’, ‘Number of fractions’ and ‘Number of patients for whom this combination has been prescribed’ are displayed. This information gives the clinician a quick view of what prescriptions have worked or been used for patients in the past.

As another example, the assistant tool provides the user with information regarding the different plans used on other patients for the same treatment site as the current patient. Details regarding treatment site, plan type (Block, Conformal or IMRT), treatment modality (X rays, electron), and accessories used are presented to the user. This information may aid the user in defining a plan for the current patient.

In another example, the assistant tool presents filters and an indication of the most used filters or combinations in the past for patients with the same tumor site. The clinician may more quickly and reliably determine which filter is going to work for a current patient. This saves time and improves efficiency in the workflow.

For yet another example, the assistant tool is invoked in an imaging task. Information on whether 2D or 3D imaging is the most preferred review method for patients with the same treatment site and plan is presented.

The historical values and any corresponding statistical information may be output in a pop-up as shown in FIG. 4. Other outputs may be used, such as a table in a document or file. In one embodiment, the information is provided as part of the task user interface 30, such as displaying the possible or previously used values adjacent to the components to select a current value. The user may hide or unhide the information, such as by selecting or unselecting the assistant icon 40 or using other input options.

In act 26, the system or software receives user input of a value for a current patient. The input value may be based on any information and/or knowledge of the user. The user may or may not review the previous values used to determine a current value. By reviewing the previously used values and associated statistical information, the user may increase the likelihood of a successful outcome or increase efficiency in selection of the values. The assistant tool acts as if consulting a colleague with historical knowledge about the current and past practices. The information may also be used to determine new possible combinations of values that may be more successful and/or to avoid combinations that may not be as successful. Where outcome information is unavailable, the historical and/or statistical information may assist the user in understanding the typical approach.

FIG. 5 shows a system for assisting an oncology workflow. The system implements the method of FIG. 1 or other methods. The system generates the user interfaces 30 of FIGS. 2-4 or other displays. The system is a personal computer, workstation, picture archiving and communication system (PACS) workstation, a network interface, part of an intranet, portable computer, or other device for assisting in medical (e.g., oncology) workflow. The system includes a user input device 50, a processor 52, a memory 54, and a display 56. These components may be provided in a same housing, a same room, or distributed. Additional, different, or fewer components may be provided.

The user input device includes a keyboard, buttons, sliders, touch screen, touch pad, position sensor, track ball, mouse, and/or other now know or later developed user input device. The user input allows a user to interact with a user interface on the display 56, such allowing a user to select or otherwise input information solicited by the user interface.

The processor 52 is an application specific integrated circuit, general processor, control processor, image processor, field programmable gate array, digital signal processor, analog circuit, digital circuit, network, server, group of processors, data path, combinations thereof, or other now known or later developed device for assisting in medical workflow. The processor 22 may be a single device or combinations of devices. The processor 22 operates pursuant to software or hardware. The processor 22 connects with the user input device 50 for receiving user selections and connects with the display 56 for generating output information.

The processor 22 is operable to receive user selections of values for respective variables associated with a prescription generation oncology task, plan generation oncology task, patient positioning oncology task, imaging selection oncology task, physicist QA oncology task, other medical tasks, or combinations thereof. The inputs received may be based, at least in part, on previous selection of an assistant icon and/or display of historical or statistical information for other patients related to the task. The processor 22 provides values for variables to assist the user. The variables are selected by the processor 22 as a function of the oncology or other task being performed by the user. For example, the processor 22 identifies the oncology task based, in part, on the user and/or any user selections of tasks.

The processor 22 receives user selections of values for any variables. The variables may be associated with the prescription generation oncology task, such as including two or more of target dose, warning dose, fraction dose and number of fractions. The variables may be associated with the plan generation oncology task, such as including two or more of plan type, treatment modality, and treatment accessories. The variables may be associated with the patient positioning oncology task, such as the variables including tolerance or image filter.

The processor 22 is operable to output example values to the display 56. The example values are for a given oncology or other task or tasks. The example values are values used for other patients with or without successful outcomes and having similar diagnostic information as a current patient. Any determination of similarity may be used. For example, similar diagnostic information includes a same size and stage of tumor as the current patient for which the prescription generation oncology task is being performed. As another example, the similar diagnostic information includes a same treatment site as the current patient for which the plan generation oncology task is being performed. In another example, the similar diagnostic information includes a same treatment site and type of treatment as the current patient for which the patient positioning oncology task is being performed.

The display 56 is a CRT, LCD, plasma screen, projector, printer, or other now known or later developed device for presenting information to a user. The display 56 may provide the user interface and any output information to the user. For example, the display 56 outputs historical and/or statistical information to the user to assist in determining values to use for a task. The display 56 provides a graph, text, chart, symbols, or other information indicating example values for the variables of a task. Statistical information, such as an indication of a number of uses of each value or combination of values or an indication of a correlation with outcome, is displayed for consideration by the user.

The memory 54 is a database or databases of tables or computerized patient records. The memory 54 stores queries, search results, data to be queried, output information, and/or other data. In one embodiment, the memory 54 stores instructions for operation of the processor 22. The instructions for implementing the processes, methods and/or techniques discussed above (e.g., for providing historical values in a medical workflow) are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for assisting an oncology workflow, the method comprising:
displaying a user interface for each of a plurality of oncology tasks, the user interface for each of the oncology tasks including one or more first tools for receiving input for a variable of the oncology task;
receiving user selection of an assistant tool for at least a first oncology task of the oncology tasks; and
displaying information for the first oncology task in response to the user selection, the information including a list of values for the variable and use frequency for each value of the list and being from records of a plurality of patients.

2. The method of claim 1 wherein displaying the user interface for each of the plurality of oncology tasks comprises displaying for prescription generation, plan generation, and patient positioning tasks.

3. The method of claim 1 wherein displaying the user interface comprises displaying for prescription generation, the variable being one of target dose, warning dose, fraction dose or number of fractions, and wherein displaying the information comprises displaying the information for the records of the patients with a corresponding size and stage of tumor as a current patient for which the prescription generation task is being performed.

4. The method of claim 1 wherein displaying the user interface comprises displaying for plan generation, the variable being one of plan type, treatment modality, or treatment accessories, and wherein displaying the information comprises displaying the information for the records of the patients with a corresponding treatment site as a current patient for which the plan generation task is being performed.

5. The method of claim 1 wherein displaying the user interface comprises displaying for patient positioning of a current patient, the variable being one of tolerance or image filter, and wherein displaying the information comprises displaying the information for the records of the patients with a corresponding tumor site and type of treatment.

6. The method of claim 1 wherein displaying the user interface comprises displaying an assistant icon, and wherein receiving the user selection comprises receiving an indication of selection of the assistant icon.

7. The method of claim 1 wherein displaying the information comprises displaying the list with an indication of a number of patients associated with each value and displaying other values for other variables associated with the first oncology task.

8. The method of claim 1 wherein displaying the information comprises:
obtaining the information as a function of the first oncology task, the records for the first oncology task of the patients with a desired outcome being included and the records of the patients without the desired outcome not being included, the values for the variable and additional variables included in the information as a function of the first oncology task.

9. The method of claim 1 wherein the displaying of the user interface is a function of a role of a user, and wherein the records and values are for patients from a same medical institution.

10. A system for assisting an oncology workflow, the system comprising:
a user input;
a display;
a processor connected with the user input and the display, the processor operable to receive user selections of values for respective variables associated with a prescription generation oncology task, plan generation oncology task, patient positioning oncology task, imaging selection oncology task, plan quality assurance oncology task, or combinations thereof, the processor operable to output to the display example values, corresponding to the oncology task, used for other patients with successful outcomes and having similar diagnostic information as a current patient;
wherein the display is operable to display the example values.

11. The system of claim 10 wherein the variables are associated with the prescription generation oncology task, the variables including two or more of target dose, warning dose, fraction dose and number of fractions, and wherein the similar diagnostic information includes a same size and stage of tumor as the current patient for which the prescription generation oncology task is being performed.

12. The system of claim 10 wherein the variables are associated with the plan generation oncology task, the variables including two or more of plan type, treatment modality, and treatment accessories, and wherein the similar diagnostic information includes a same treatment site as the current patient for which the prescription generation oncology task is being performed.

13. The system of claim 10 wherein the variables are associated with the patient positioning oncology task, the variables including tolerance or image filter, and wherein the similar diagnostic information includes a same treatment site and type of treatment as the current patient for which the patient positioning oncology task is being performed.

14. The system of claim 10 wherein the user selection is responsive to selection of an assistant icon displayed with the oncology task.

15. The system of claim 10 wherein the example values are displayed with an indication of a number of patients associated with each value.

16. The system of claim 10 wherein the processor is operable to include the variables as a function of the oncology task.

17. The system of claim 10 wherein the processor is operable to identify the oncology task based on an identity of a user.

18. In a computer readable storage medium having stored therein data representing instructions executable by a programmed processor for providing historical values in a medical workflow, the storage medium comprising instructions for:
outputting statistical information for possible values in a current medical task as a function of past use of the possible values for other patients; and
receiving user input of an actual value to be used for a current patient.

19. The computer readable storage medium of claim 18 wherein outputting statistical information comprises outputting a number of past uses for each of the possible values, the statistical information and possible values being output for each of a plurality of variables associated with the current medical task.

20. The computer readable storage medium of claim 18 further comprising instructions for selecting the other patients as a function of diagnostic information similarity with the current patient.
